(19) United States
(12) Patent Application Publication
(10) Pub. No.: US 2015/0165154 A1
(43) Pub. Date: Jun. 18, 2015

(54) TUBING-ILLUMINATION APPARATUS

(71) Applicant: Glow Tube International, LLC, Hays, KS (US)

(72) Inventors: Sandra M. Watchous, Hays, KS (US); Thu Ngoc Mai, Hays, KS (US); Clifford M. Miyamoto, Arroyo Grande, CA (US); Yung Yip, Afton, MN (US); Daniel P. Stubbs, Marine on St. Croix, MN (US)

(21) Appl. No.: 14/531,061

(22) Filed: Nov. 3, 2014

Related U.S. Application Data
(60) Provisional application No. 61/962,247, filed on Nov. 4, 2013.

(51) Int. Cl. A61M 16/08; F21V 33/00; F21V 23/02; F21V 23/00; A61M 16/10; F21V 21/088

(52) U.S. Cl. CPC A61M 16/0875; A61M 16/1005; F21V 21/088; F21V 23/02; F21V 23/003; F21V 33/0068; A61M 2205/587; F21W 2131/20

(57) ABSTRACT

A tubing-illumination apparatus for illuminating oxygen supply tubing. The apparatus includes an electroluminescent wire that is wrapped around an oxygen supply tubing in a spiraling configuration to extend along the length thereof. Clips with a minor and a major channel extending therethrough are provided for retaining the arrangement of the wire and the tubing. The minor channel receives and retains the wire and the major channel receives and retains the tubing in a parallel, side-by-side relation. The electroluminescent wire provides illumination of the tubing continuously along the length thereof. The clips are removable from the tubing to enable replacement of the tubing. The electroluminescent wire may be coupled to a control module such that the wire may be illuminated in relation to operational conditions of an oxygen supply apparatus.

### CROSS-REFERENCE TO RELATED APPLICATIONS

This application claims the benefit of U.S. Provisional Patent Application No. 61/962,247 filed Nov. 4, 2013 and titled ILLUMINATION OF OXYGEN SUPPLY TUBING WITH EXTERNAL APPLICATION OF ELECTROLUMINESCENT WIRING the disclosure of which is hereby incorporated herein in its entirety by reference.

### BACKGROUND

Patients with chronic obstructive pulmonary disease (COPD) or other ailments requiring long-term high-oxygen concentration therapy often receive an oxygen supply through an elongated oxygen tube attached to a stationary oxygen source. The oxygen supply tube may be up to fifty feet in length to provide the patient with ease of moving around their home or dwelling without needing to move the oxygen source. The tubes can thus pose a potential tripping and entanglement hazard to the patient and other occupants of the home or dwelling. The tube may also become crushed or pinched by the patient or others standing on or placing objects on the tubing and thus restricting the flow of oxygen to the patient.

It is therefore desirable to increase the visibility of the oxygen supply tubing, especially in low-light and darkened environments. Several previous embodiments have been proposed to illuminate oxygen supply tubing, these include painting or attaching phosphorescent (e.g. glow-in-the-dark) materials or lights onto the tube. These previous attempts are encumbered by several shortcomings: the intensity of the illumination emitted by the phosphorescent material diminishes rapidly and cannot produce sufficient illumination after a few hours; and strings of lights coupled to the tubing only provide intermittent illumination along the length of the oxygen supply tubing.

What is needed is a way to provide a continuous and constant illumination along the entire length of the oxygen supply tubing.

### SUMMARY

Embodiments of the invention are defined by the claims below, not this summary. A high-level overview of various aspects of the invention is provided here to introduce a selection of concepts that are further described in the Detailed-Description section below. This summary is not intended to identify key features or essential features of the claimed subject matter, nor is it intended to be used in isolation to determine the scope of the claimed subject matter. In brief, this disclosure describes, among other things, a tubing-illumination apparatus that provides continuous illumination along the length of a section of tubing.

The tubing comprises available oxygen supply tubing with an electroluminescent wire wrapped around the exterior thereof. The electroluminescent wire is wrapped around the exterior of the oxygen supply tubing at approximately two revolutions per every eighteen inches. Clips are coupled to the electroluminescent wire and to the oxygen supply tubing to retain the arrangement of the wire on the tubing. The clips are removable from the oxygen supply tubing to enable replacement of the tubing. The tubing is connected at a first end to an oxygen supply apparatus and at a second end to an oxygen provision apparatus, such as a nasal cannula or mask that is wearable by the patient. The electroluminescent wire is coupled to a power supply that is separate from or integrated with the oxygen supply apparatus.

The clips include a longitudinally elongate form with a minor channel and a major channel extending longitudinally therethrough and open to an edge thereof. The minor channel is positioned at a base of the major channel and is open thereto. The minor channel is dimensioned to receive the electroluminescent wire therein and to provide a friction-fit with the wire. The major channel is dimensioned to receive the oxygen supply tubing and to provide a friction-fit with the tubing. Opposite longitudinal ends of the clip are chamfered or rounded to resist catching on objects or surfaces and to resist injury to persons stepping on the clips.

### DESCRIPTION OF THE DRAWINGS

Illustrative embodiments of the invention are described in detail below with reference to the attached drawing figures, and wherein:

FIG. 1 is an illustrative view of a patient receiving oxygen from an oxygen supply unit via a section of tubing as known in the art;

FIG. 2 is perspective view of a portion of a tubing-illumination apparatus depicted in accordance with an embodiment of the invention;

FIG. 3 is a side view of a clip coupled to a tubing and to a light-producing device in accordance with an embodiment of the invention;

FIG. 4 is an end view of the clip of FIG. 3 with the tubing and light-producing device removed; and

FIG. 5 is a perspective view of another clip useable with a tubing-illumination apparatus in accordance with embodiments of the invention.

### DETAILED DESCRIPTION

The subject matter of select embodiments of the invention is described with specificity herein to meet statutory requirements. But the description itself is not intended to necessarily limit the scope of claims. Rather, the claimed subject matter might be embodied in other ways to include different components, steps, or combinations thereof similar to the ones described in this document, in conjunction with other present or future technologies. Terms should not be interpreted as implying any particular order among or between various steps herein disclosed unless and except when the order of individual steps is explicitly described. The terms “about” or “approximately” as used herein denote deviations from the exact value by +/−10%, preferably by +/−5% and/or deviations in the form of changes that are insignificant to the function.

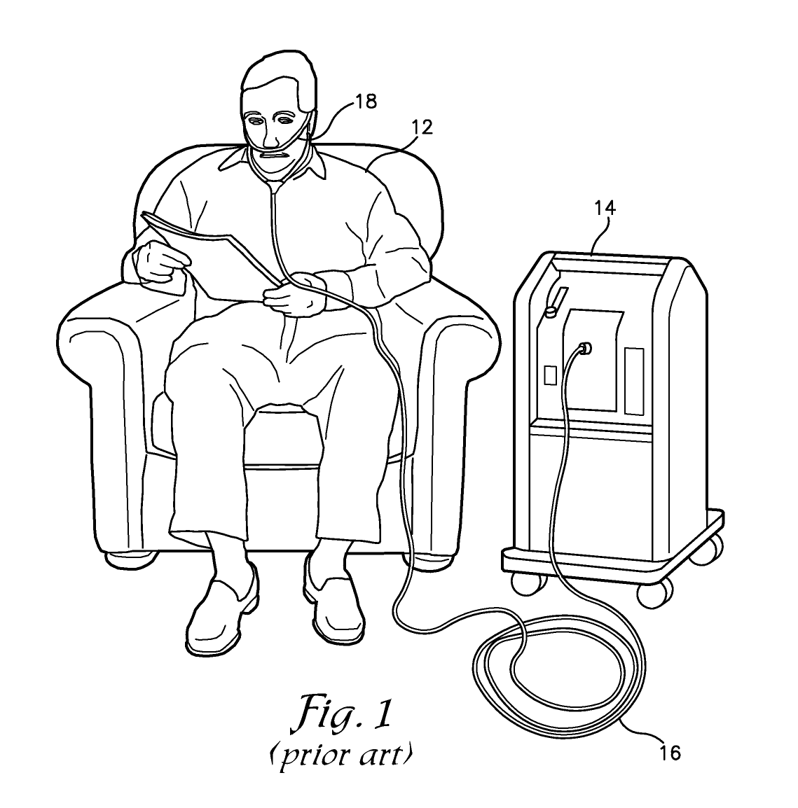

FIG. 1 depicts a patient 12, such as a patient being treated for chronic obstructive pulmonary disease (COPD) among other ailments, that is receiving a therapy requiring provision of one or more gases, e.g. oxygen, for inhalation thereby as known in the art. The patient 12 is provided with an oxygen supply apparatus 14 that supplies the oxygen in a desired concentration. A section of tubing 16 is connected to the oxygen supply apparatus 14 and includes a gas-provision attachment 18 at an opposite end. The gas-provision attachment 18 may include, for example, a nasal cannula, an oxygen mask, or a rebreather, among others forms. The section of tubing 16 may be of any desired length to enable the patient 12 freedom of movement without the need for moving the oxygen supply apparatus 14. In embodiments, the tubing 16 is provided in twenty-five or fifty foot lengths that can be coupled end-to-end to extend the overall length.

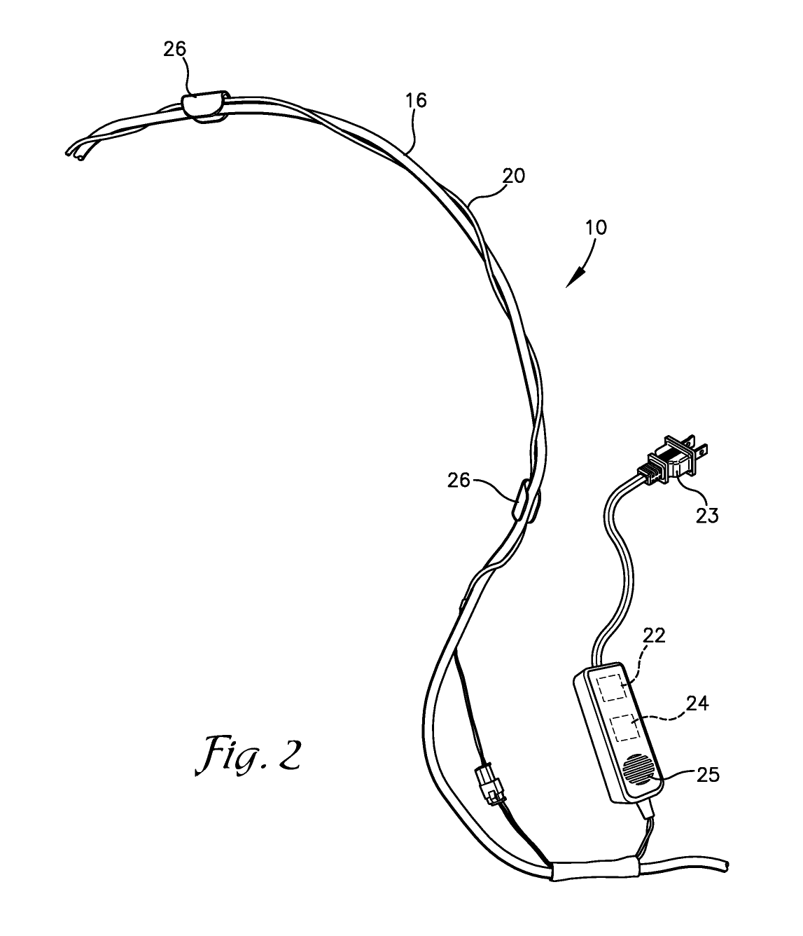

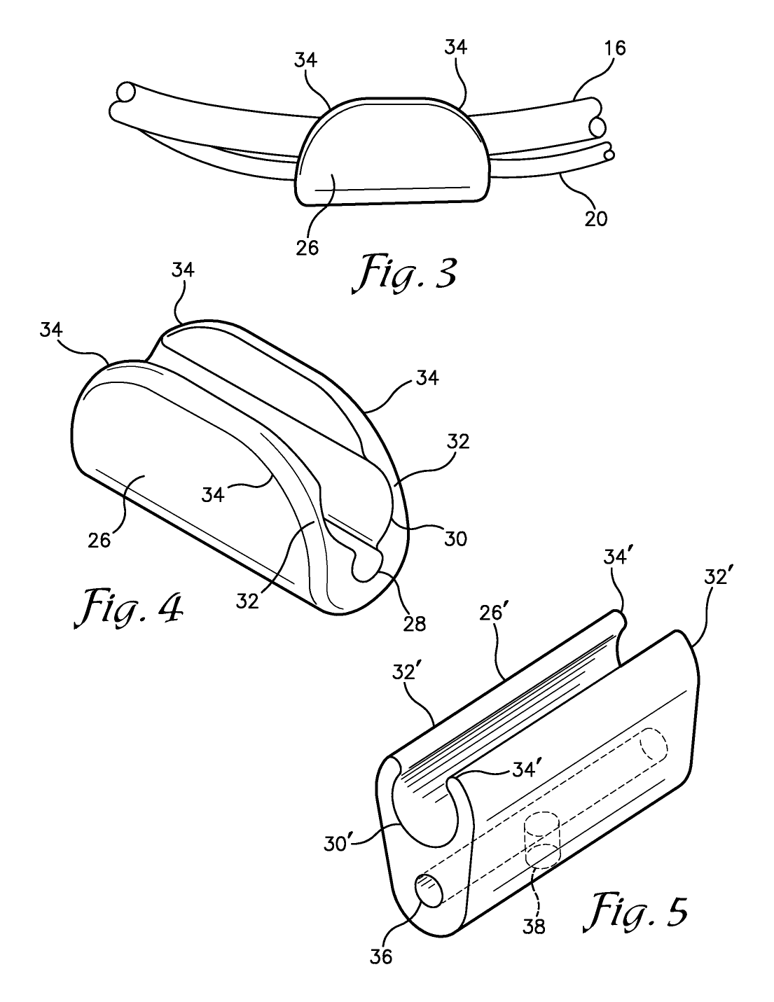

With reference now to FIGS. 2-5, a tubing-illumination apparatus 10 is described in accordance with an embodiment of the invention. Embodiments of the invention are described herein with respect to provision of oxygen or high-concentration oxygen, but such is not intended to so limit application of embodiments of the invention. The apparatus 10 may be employed tubing or hoses for use in provision of other gases, liquids, or solids in the medical or other fields in which illumination of the tubing is desired. Embodiments of the invention might also be configured for use with items other than the tubing 16, such as wires, cables, equipment, furniture, or the like.

With reference to FIG. 2, the apparatus 10 includes a light-producing device 20 that has an elongate, wire-, tube- or rope-like form. The light-producing device 20 preferably comprises an electroluminescent wire, such as EL wire available from KITELIGHT PRODUCTS of Rotterdam, Netherlands, but may comprise one or more of a light guide, a phosphorescent (glow-in-the-dark) member, a fluorescent member, and light-emitting diodes (LEDs), among others. Electroluminescent wires as known in the art include an inner core wire surrounded by a phosphor compound layer and one or more exterior coating layers. One or more lead wires are disposed between the phosphor compound layer and the exterior coating layers. When electrified, the lead wires and the inner core wire create an electric field that excites the electrons of the phosphor compound and causes the emission of light therefrom. One or more of the exterior coating layers may be colored to provide a desired color to the emitted light. The emitted light is produced about the entire circumference of the wire and along the entire length thereof.

The light-producing device 20 is configured for continuous production of light over at least a 12-15 hour period. The device 20 is also preferably constructed to withstand a variety of conditions that may be faced during use. These may include the device 20 being walked on, stepped on, and rolled over by the patient 12, support staff, equipment, wheelchairs, and the like, as well as stretching and bending as the device 20 is coiled up or pulled around corners, furniture, or the like. The light-producing device 20 is also preferably non-reactive and non-flammable so as to be safe for use around concentrations of oxygen and/or other gases.

The light-producing device 20 includes a power supply 22 that is electrically coupleable to the device 20. The power supply 22 may include one or more transformers, inverters or the like as needed to provide an appropriate power output for use by the device 20. The power supply 22 can include one or more batteries or can couple to a local power grid via, for example, a plug 23. In one embodiment, the power supply 22 is integrated with the oxygen supply apparatus 14.

The light-producing device 20 may also include a control module 24. The control module 24 can be integral with the power supply 22 and/or with the oxygen supply apparatus 14 and may interface with one or more sensors in the oxygen supply apparatus 14 or in-line with the oxygen flow. The control module 24 is configured to control illumination of the light-producing device 20, e.g. an on/off state and/or an intensity of the emitted light. The control module 24 may cause the light-producing device 20 to illuminate or blink in a pattern relative to an operational state of the oxygen supply apparatus 14 and/or based on a time of day or light conditions of the environment. For example, the light-producing device 20 may be constantly illuminated during normal functioning of the oxygen supply apparatus 14 but may flash in a predetermined pattern when a fault condition is sensed, e.g. when the tubing 16 is obstructed or when the apparatus 14 is in a fault condition. The light-producing device 20 might also be turned off during daytime hours or when environmental lighting conditions provide at least a predetermined level of illumination. The control module 24 may also include or be in communication with an audible-alert module 25 that can be employed to produce audible alerts in association with the operating conditions of the oxygen supply apparatus 14.

With continued reference to FIGS. 2-4, the apparatus 10 also includes a plurality of clips 26 that couple the light-producing device 20 to the tubing 16. The clips 26 are configured to releasably couple the light-producing device 20 to the tubing 16 such that the tubing 16 can be periodically replaced. The clips 26 can releasably or fixedly couple to the light-producing device 20. As depicted in FIGS. 2-4, the clips 26 are elongated to extend in a longitudinal direction relative to the tubing 16 and light-producing device 20. A minor channel 28 and a major channel 30 are provided in the clips 26 extending generally parallel to one another and in the longitudinal direction. Both channels 28, 30 include a generally C-shaped cross-sectional profile. The C-shape of the minor channel 28 is open to the major channel 30 generally along the apex of the C-shape of the major channel 30. The C-shape of the major channel 30 is open to the edge of the clip 26 and to the environment.

Both channels 28, 30 are dimensioned provide a firm, fixed grip on the respective light-producing device 20 and tubing 16 such that the clips 26 do not easily slide along the device 20 or tubing 16. The clips 26 may be configured to receive the respective light-producing device 20 and the tubing 16 in a friction-fit manner; the clip 26 may partially flex and/or the light-producing device 20 and tubing 16 may be partially compressed when engaged with the channels 28, 30. In another embodiment, one or more glues, resins, bonding agents, or adhesives (hereinafter referred to collectively as adhesives) may be used to fixedly couple the clips 26 to one or both of the light-producing device 20 and the tubing 16.

The channels 28, 30 of the clips 26 form a sidewall 32 or flange on each side of the clip 26. Edges or corners 34 of the sidewalls 32 may be chamfered, rounded, or truncated to remove sharp corners and edges from the clips 26. As such, risks of the clips 26 damaging the tubing 16 or injuring the patient 12 or other person when the clips 26 are stepped on or otherwise contacted are reduced.

Although a particular configuration of the clips 26 is described herein, other configurations may be employed without departing from the scope of embodiments of the invention. For example, as depicted in FIG. 5, a clip 26′ includes a major channel 30′ configured to receive the tubing 16 and a duct 36 through which the light-producing device 20 is insertable. The clip 26′ may include an orifice 38 that is in communication with the duct 36 to enable injection of an adhesive into the duct 36 after insertion of the light-producing device 20 therein to fixedly couple the clip 26′ to the light-producing device 20. The sidewalls 32′ of the clip 26′ also do not include rounded or chamfered corners 34′.

With continued reference to FIGS. 1-4, use and operation of the tubing-illumination apparatus 10 is described in accordance with an embodiment of the invention. A section of the tubing 16 is obtained in a desired length. The tubing 16 may be provided in shorter lengths of, for example, twenty-five feet, that are coupleable end-to-end to extend the tubing 16 to a desired length. The light-producing device 20 is wrapped around the exterior of the tubing 16 in a spiraling manner, as depicted in FIG. 2.

The light-producing device 20 is preferably wrapped about two times around the tubing 16 per every approximately eighteen inches of tubing 16, but a greater or lesser number of wraps may be employed. Two wraps per eighteen inches maintains the light-producing device 20 in sufficiently close side-by-side relation to the tubing 16 so as not to produce additional snagging or entanglement hazards. Such a configuration also provides sufficient illumination of the tubing 16 without requiring extensive additional lengths of the light-producing device 20.

The light-producing device 20 may be provided in lengths sufficient to accommodate two wraps per eighteen inches of tubing 16, or other configurations, for pre-selected lengths of tubing 16, e.g. lengths of the light-producing device 20 that are slightly longer than twenty-five or fifty feet may be provided to accommodate standard twenty-five or fifty foot lengths of tubing 16. Additional lengths of the light-producing device 20 might also be coupleable end-to-end to increase the overall length thereof as desired.

The clips 26 are first coupled to the light-producing device 20 by inserting the light-producing device 20 through the major channel 30 and into the minor channel 28. The tubing 16 is then inserted into the major channel 20. The clips are preferably spaced apart along the length of the tubing 16 at about eighteen inch intervals, however other spacing may be employed.

The tubing 16 can be coupled to the oxygen supply apparatus 14 and the gas-provision attachment provided to the patient 12 to receive oxygen therapy. The light-producing device 20 is coupled to the power supply 22 and may be coupled to the control module 24 to provide illumination thereof.

Accordingly, substantially the full length of the tubing 16 is illuminated by the light-producing device 20 and is thus easily visible when lying on the floor or stretched about the patient's living space. The patient 12 and any other occupants of the living space may thus more easily see and recognize the tubing 16 as such and avoid stepping on, tripping over, or becoming entangled therewith. Additionally, when the control module 24 is employed, the patient 12 or a caregiver may also be alerted to fault conditions or problems with the oxygen supply apparatus 14 by flashing or blinking of the light-producing device 20. The audible-alert module 25 may also provide audible alerts associated with such fault conditions.

The tubing 16 may have a relatively short duty cycle or time period within which the tubing 16 is useable, such as about one month or thirty days. As such, the clips 26 can be removed or uncoupled from the tubing 16 to allow the light-producing device 20 to be unwrapped or otherwise removed from the tubing 16 to enable replacement of the tubing 16 with a new section of tubing 16.

Many different arrangements of the various components depicted, as well as components not shown, are possible without departing from the scope of the claims below. Embodiments of the technology have been described with the intent to be illustrative rather than restrictive. Alternative embodiments will become apparent to readers of this disclosure after and because of reading it. Alternative means of implementing the aforementioned can be completed without departing from the scope of the claims below. Identification of structures as being configured to perform a particular function in this disclosure and in the claims below is intended to be inclusive of structures and arrangements or designs thereof that are within the scope of this disclosure and readily identifiable by one of skill in the art and that can perform the particular function in a similar way. Certain features and sub-combinations are of utility and may be employed without reference to other features and sub-combinations and are contemplated within the scope of the claims.

What is claimed is:

1. A tubing illumination apparatus comprising:
an elongate light-producing device configured to be wrapped around an exterior of a section of tubing and to extend substantially the length of the tubing;
a plurality of clips, each clip coupling to the light-producing device and coupleable to the section of tubing and in a spaced apart arrangement along the length of the tubing; and
a power supply coupled to a first end of the light-producing device.

2. The tubing illumination apparatus of claim 1, wherein each clip includes a minor channel and a major channel, the minor channel receiving the light-producing device and the major channel configured for receiving the tubing.

3. The tubing illumination apparatus of claim 2, wherein the minor channel and the major channel extend in a substantially parallel orientation and have generally C-shaped cross-sectional profiles, and wherein the C-shaped profile of the minor channel is open to the major channel and the C-shaped profile of the major channel is open to the environment.

4. The tubing illumination apparatus of claim 2, wherein the light-producing device is received by the minor channel through the major channel.

5. The tubing illumination apparatus of claim 1, wherein one or more of the clips includes a duct and a channel extending in parallel along the length of the clip, the duct receiving the light-producing device and the channel configured to receive the tubing.

6. The tubing illumination apparatus of claim 5, wherein one or more of the clips includes an orifice in communication with the duct, and wherein an adhesive is disposed in the duct via the orifice to secure the clip to the light-producing device.

7. The tubing illumination apparatus of claim 1, wherein the light-producing device comprises an electroluminescent wire.

8. The tubing illumination apparatus of claim 1, further comprising:
a control module in electrical communication with the light-producing device and configured to control illumination of the light-producing device.

9. The tubing illumination apparatus of claim 8, wherein the control module provides a visual signal associated with an operational condition of a device to which the section of tubing is coupled by changing the illumination of the light-producing device.

10. The tubing illumination apparatus of claim 1, further comprising:
the section of tubing; and
an oxygen supply apparatus operably coupled to the section of tubing to provide oxygen therapy to a patient via the tubing.

11. The tubing illumination apparatus of claim 1, wherein the clips are removable from the section of tubing to enable replacement of the section of tubing with a second section of tubing.

12. The tubing illumination apparatus of claim 1, wherein the light-producing device includes a plurality of light-producing devices coupled end-to-end.

13. A tubing-illumination apparatus for illuminating oxygen supply tubing, the apparatus comprising:
an oxygen supply apparatus;
a section of tubing operably coupled to the oxygen supply apparatus to receive a supply of oxygen;
an elongate light-producing device wrapped around the section of tubing and extending substantially the length of the tubing; and
a plurality of clips, each clip coupling to the light-producing device and to the section of tubing and being spaced apart along the length of the tubing.

14. The tubing illumination apparatus of claim 13, wherein each clip includes a minor channel and a major channel, the minor channel receiving the light-producing device and the major channel receiving the tubing.

15. The tubing illumination apparatus of claim 13, wherein the light-producing device comprises an electroluminescent wire.

16. The tubing illumination apparatus of claim 13, further comprising:
a control module in electrical communication with the light-producing device and with the oxygen supply apparatus, the control module operable to control illumination of the light-producing device to signal an operational condition of the oxygen supply apparatus.

17. A method for illuminating a section of oxygen supply tubing;
providing a section of oxygen supply tubing;
wrapping an electroluminescent wire around the outer surface of the tubing in a spiraling configuration extending substantially the length of the tubing;
coupling a plurality of clips to the electroluminescent wire and the tubing at spaced apart locations along the length of the tubing;
coupling a first end of the tubing to an oxygen supply apparatus and a second end of the tubing to an oxygen provision apparatus useable by a patient to receive the oxygen; and
connecting the electroluminescent wire to a power source to illuminate the electroluminescent wire.

18. The method of claim 17, further comprising:
electrically coupling the electroluminescent wire to a second electroluminescent wire in an end-to-end orientation to extend the length of the electroluminescent wire.

19. The method of claim 17, wherein each clip includes a minor channel and a major channel extending longitudinally along the length of the clip, and further comprising:
disposing the electroluminescent wire in the minor channel; and
disposing the tubing in the major channel, the tubing being retained in the major channel by a friction-fit engagement.

20. The method of claim 17, wherein when the tubing reaches and end of a duty cycle, the method further comprising:
removing the tubing from each of the plurality of clips;
unwrapping the electroluminescent wire from around the tubing;
providing a second section of tubing;
wrapping the electroluminescent wire around the outer surface of the second section of tubing;
coupling the plurality of clips to the second section of tubing at spaced apart locations along the length of the second section of tubing.
